(19) United States
(12) Patent Application Publication
(10) Pub. No.: US 2025/0213161 A1
(43) Pub. Date: Jul. 3, 2025

(54) Multifunction Electrode Pad With Integrated 12-Lead ECG Acquisition

(71) Applicant: CB Innovations, LLC, Carlsbad, CA (US)

(72) Inventors: Christian McClung, Rancho Santa Fe, CA (US); Stephen Dunphy, Carlsbad, CA (US)

(73) Assignee: CB Innovations, LLC, Carlsbad, CA (US)

(21) Appl. No.: 19/085,558

(22) Filed: Mar. 20, 2025

Related U.S. Application Data
(63) Continuation-in-part of application No. 18/384,872, filed on Oct. 29, 2023.
Application No. 18/384,872 is a continuation-in-part of application No. 17/665,003, filed on Feb. 4, 2022.
Application No. 17/665,003 is a continuation-in-part of application No. 16/812,330, filed on Mar. 8, 2020, now Pat. No. 11,896,393.
Application No. 16/812,330 is a continuation-in-part of application No. 15/990,651, filed on May 27, 2018, now Pat. No. 10,881,313.
Application No. 15/990,651 is a continuation of application No. 15/853,578, filed on Dec. 22, 2017, now Pat. No. 9,986,929.
(63) Continuation-in-part of application No. 17/106,125, filed on Nov. 29, 2020.
Application No. 17/106,125 is a division of application No. 15/904,411, filed on Feb. 25, 2018, now Pat. No. 10,893,818.
Application No. 15/904,411 is a continuation-in-part of application No. 15/853,578, filed on Dec. 22, 2017, now Pat. No. 9,986,929.
(60) Provisional application No. 62/825,018, filed on Mar. 27, 2019.
(60) Provisional application No. 62/465,752, filed on Mar. 1, 2017.
(60) Provisional application No. 62/530,144, filed on Jul. 8, 2017.
(60) Provisional application No. 63/147,191, filed on Feb. 8, 2021.
(60) Provisional application No. 62/819,025, filed on Mar. 15, 2019.
(60) Provisional application No. 63/573,462, filed on Apr. 3, 2024.
(60) Provisional application No. 63/421,569, filed on Nov. 2, 2022.

(51) Int. Cl. A61B 5/282; A61B 5/00; A61B 5/02; A61B 5/0205; A61B 5/021; A61B 5/024; A61B 5/08; A61B 5/256; A61B 5/257; A61B 5/259; A61B 5/271; A61B 5/28; A61B 5/30; A61B 5/308; A61B 5/316; A61B 5/318; A61B 5/333; A61B 5/338

(52) U.S. Cl. CPC A61B 5/282; A61B 5/259; A61B 5/303; A61B 5/6841; A61B 5/02; A61B 5/0205; A61B 5/021; A61B 5/024; A61B 5/08; A61B 5/0803; A61B 5/256; A61B 5/257; A61B 5/271; A61B 5/28; A61B 5/308; A61B 5/316; A61B 5/318; A61B 5/333; A61B 5/338; A61B 2505/01; A61B 2562/02; A61B 2562/0209; A61B 2562/04; A61B 2562/046; A61B 2562/222; A61B 2562/227

(57) ABSTRACT

A multifunction electrode pad with integrated 12-Lead ECG acquisition is disclosed herein. The electrodes are incorporated into the two defibrillation electrode pads. Each of the sternum defibrillation pad and the apex defibrillation pad is composed of an electrical conductive plates in a foam tape substrate with a hydrogel layer. Each of the sternum pad and the apex pad has defibrillation electrode pad conductor surface has an area ranging from 80 cm³ to 90 cm³.

### CROSS REFERENCES TO RELATED APPLICATIONS

The Present Application claims priority to U.S. Provisional Patent Application No. 63/573,462, filed on Apr. 3, 2024, and the Present Application is a continuation-in-part application of U.S. patent application Ser. No. 18/384,872, filed on Oct. 29, 2023, which claims priority to U.S. Provisional Patent Application No. 63/421,569, filed on Nov. 2, 2022, now expired, and U.S. patent application Ser. No. 18/384,872 is a continuation-in-part application of U.S. patent application Ser. No. 17/665,003, filed on Feb. 4, 2022, which claims priority to U.S. Provisional Patent Application No. 63/147,191, filed on Feb. 8, 2021, now expired, which is a continuation-in-part application of U.S. patent application Ser. No. 16/812,330, filed on Mar. 8, 2020, now U.S. Pat. No. 11,896,393 issued on Feb. 13, 2024, which claims priority to U.S. Provisional Patent Application No. 62/819,025 filed on Mar. 15, 2019, now expired, and U.S. Provisional Patent Application No. 62/825,018 filed on Mar. 27, 2019, now expired, and U.S. patent application Ser. No. 16/812,330 is a continuation-in-part application of U.S. patent application Ser. No. 15/990,651, filed on May 27, 2018, now U.S. Pat. No. 10,881,313, issued on Jan. 5, 2021, which is a continuation application of U.S. patent application Ser. No. 15/853,578, filed on Dec. 22, 2017, now U.S. Pat. No. 9,986,929, issued on Jun. 5, 2018, which claims priority to U.S. Provisional Patent Application No. 62/465,752, filed on Mar. 1, 2017, now expired, and also claims priority to 62/530,144, filed on Jul. 8, 2017, now expired, each of which is hereby incorporated by reference in its entirety, and U.S. patent application Ser. No. 17/665,003 is also a continuation-in-part application of U.S. patent application Ser. No. 17/106,125, filed on Nov. 29, 2020, which is a divisional of U.S. patent application Ser. No. 15/904,411, filed on Feb. 25, 2018, now U.S. Pat. No. 10,893,818, issued on Jan. 19, 2021, which is a continuation-in-part application of U.S. patent application Ser. No. 15/853,578, filed on Dec. 22, 2017, now U.S. Pat. No. 9,986,929, issued on Jun. 5, 2018, each of which is hereby incorporated by reference in its entirety

### STATEMENT REGARDING FEDERALLY SPONSORED RESEARCH OR DEVELOPMENT

Not Applicable

### BACKGROUND OF THE INVENTION

### Field of the Invention

The present invention generally relates to ECG devices.

### Description of the Related Art

Defibrillation pads are an essential component of defibrillators and are used to deliver a shock to the heart in case of sudden cardiac arrest. Given the aging population, increase in comorbidities including but not limited to obesity, hypertension and diabetes, and an increase in the incidence of sudden cardiac death as well as atrial fibrillation is expected. The increase in atrial fibrillation incidence in the US population is being felt in the emergency department as well. The inventors note an increased personal experience in performing electrical cardioversion for atrial fibrillation. The use of disposable defibrillation pads is directly related to the incidence of sudden cardiac death/arrest and risk of arrythmia, such as atrial fibrillation, that are often treated with electrical cardioversion.

### BRIEF SUMMARY OF THE INVENTION

The device of the present invention combines 12-lead cardiac electrodes with two multifunction defibrillation pads composed of electrically conductive plates in a foam tape substrate with hydrogel layer. It is intended for use in specific medical conditions where EKG acquisition, cardiac monitoring, rhythm interpretation, cardiopulmonary resuscitation, defibrillation and/or cardiac pacing may be required. It is to be used by prescription only and by trained medical professionals. The device has the ability to transfer energy from a cardiac defibrillator or pacer to the body of a patient for the purpose of defibrillation, synchronized cardioversion, and/or transcutaneous cardiac pacing. The device has the unique ability to also transfer 12-lead EKG signals from the body of the patient to a cardiac defibrillator/monitor or pacer for 12-lead EKG acquisition.

One aspect of the present invention is a multifunction electrode pad with integrated 12-Lead ECG acquisition. The apparatus comprises a sternum pad and an apex pad. The sternum pad comprises a body with a first defibrillation pad, a first electrode, a second electrode, and a third electrode. The sternum pad also comprises a right arm member having a fourth electrode and a line to the body. The apex pad comprises a body with a second defibrillation pad, a fifth electrode, a sixth electrode, a seventh electrode and an eighth electrode. The apex pad also comprises a left arm member having a ninth electrode and a lower left member having a tenth electrode. Each of the first defibrillation pad and the second defibrillation pad is composed of an electrical conductive plates in a foam tape substrate with a hydrogel layer.

Another aspect of the present invention is a multifunction electrode pad with integrated 12-Lead ECG acquisition. The apparatus comprises a sternum pad and an apex pad. The sternum pad comprises a body with a first defibrillation pad and a first elongation section. The first elongation section comprises a first electrode member with a first electrode, a second electrode member with a second electrode, a first expansion section between the first electrode member and the second electrode member, and a third electrode. The sternum pad also comprises a right arm member having a fourth electrode and a first line to the body. The apex pad comprises a body with a second defibrillation pad and a second elongation section. The second elongation section comprises a third electrode member with a fifth electrode, a fourth electrode member with a sixth electrode, a second expansion section between the third electrode member and the fourth electrode member, a fifth electrode member with a seventh electrode, a third expansion section between the fourth electrode member and the fifth electrode member, a sixth electrode member with an eighth electrode, a fourth expansion section between the fifth electrode member and the sixth electrode member. The apex pad also comprises a left arm member having a ninth electrode with a second line to the body, and a lower left member having a tenth electrode and a third line to the body. Each of the first defibrillation pad and the second defibrillation pad is composed of an electrical conductive plates in a foam tape substrate with a hydrogel layer.

Having briefly described the present invention, the above and further objects, features and advantages thereof will be recognized by those skilled in the pertinent art from the following detailed description of the invention when taken in conjunction with the accompanying drawings.

### BRIEF DESCRIPTION OF THE SEVERAL VIEWS OF THE DRAWINGS

FIG. 1A is a top plan view of an apex pad of the multifunction electrode pad.

FIG. 1B is a detailed section of the apex pad of FIG. 1A.

FIG. 2 is a bottom plan view of the apex pad of FIG. 1.

FIG. 3 is a front elevation view of the apex pad of FIG. 1.

FIG. 4 is a rear elevation view of the apex pad of FIG. 1.

FIG. 5 is a side elevation view of the apex pad of FIG. 1.

FIG. 6 is a side elevation view of the apex pad of FIG. 1.

FIG. 7 is a top plan view of a sternum pad of the multifunction electrode pad.

FIG. 8 is a bottom plan view of the sternum pad of FIG. 7.

FIG. 9 is a front elevation view of the sternum pad of FIG. 7.

FIG. 10 is a rear elevation view of the sternum pad of FIG. 7.

FIG. 11 is a side elevation view of the sternum pad of FIG. 7.

FIG. 12 is a side elevation view of the sternum pad of FIG. 7.

FIG. 13 is an illustration of the apex pad on a person.

FIG. 14 is an illustration of the sternum pad on a person.

FIG. 15 is an illustration of the multifunction electrode pad with integrated 12-lead ECG acquisition device on a person.

FIG. 16 is an illustration of an apex liner from the front.

FIG. 17A is an illustration of an apex liner from the rear.

FIG. 17B is an illustration of the foam portions of an apex pad from the rear.

FIG. 18 is an illustration of internal circuitry of the apex liner.

FIG. 19 is an illustration of a sternum liner from the front.

FIG. 20A is an illustration of a sternum liner from the rear.

FIG. 20B is an illustration of the foam portions of a sternum pad from the rear.

FIG. 21 is an illustration of internal circuitry of the sternum liner.

FIG. 22 is an illustration of another embodiment of the multifunction electrode pad with integrated 12-lead ECG acquisition device on a person.

FIG. 23 is a top plan view of a 3-lead component.

FIG. 24 is an exploded view of a 3-lead component.

### DETAILED DESCRIPTION OF THE INVENTION

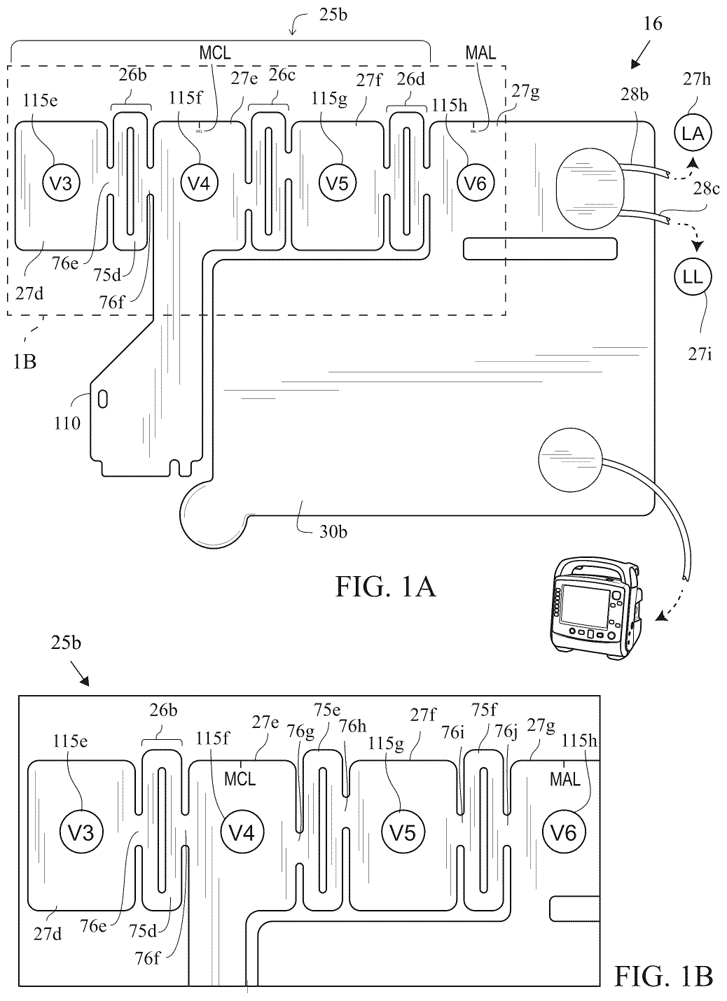

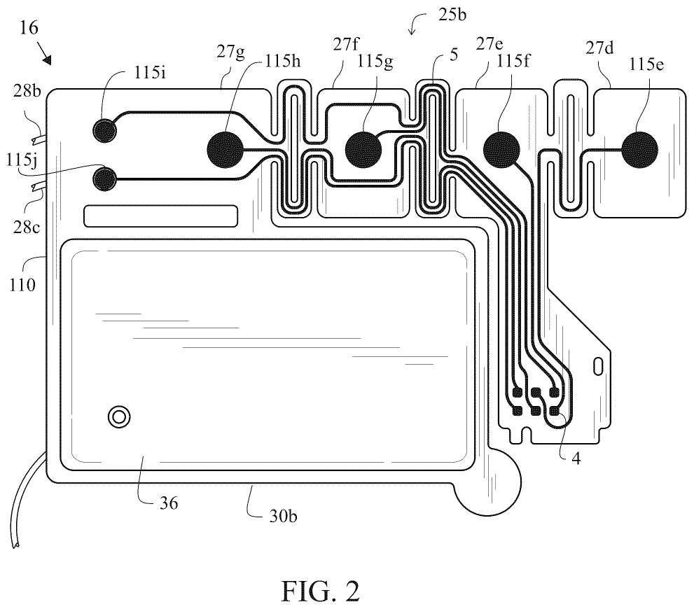

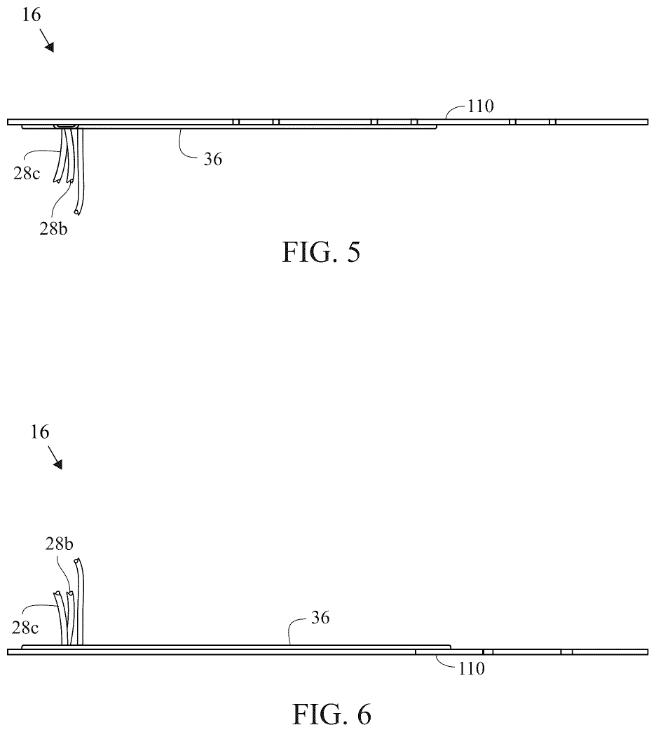

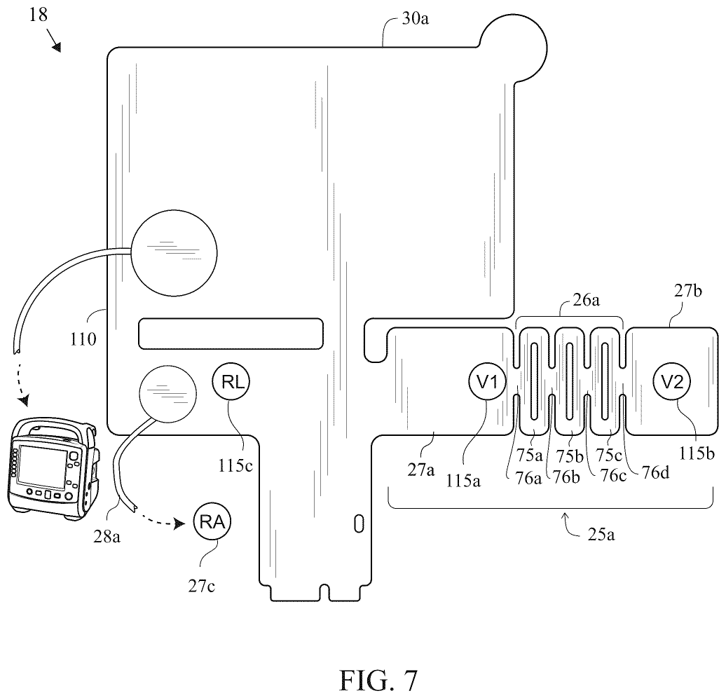

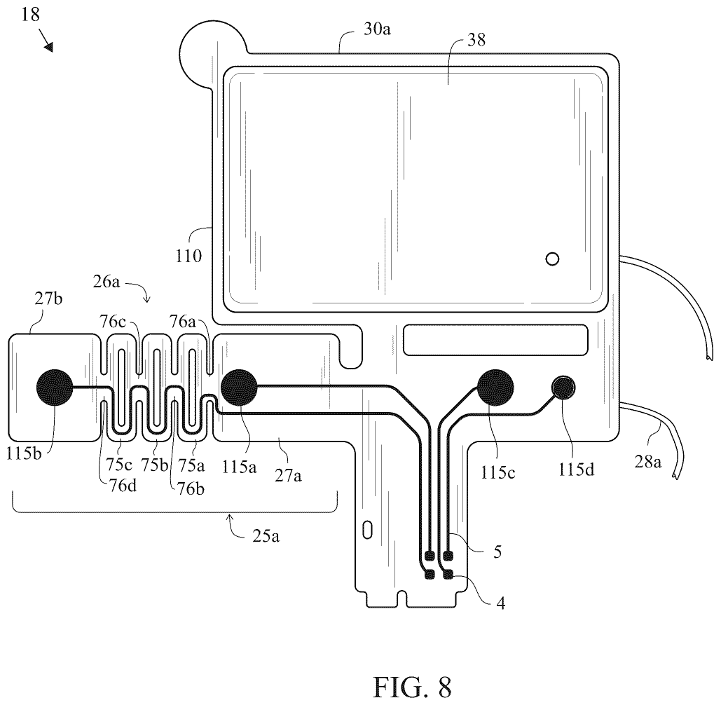

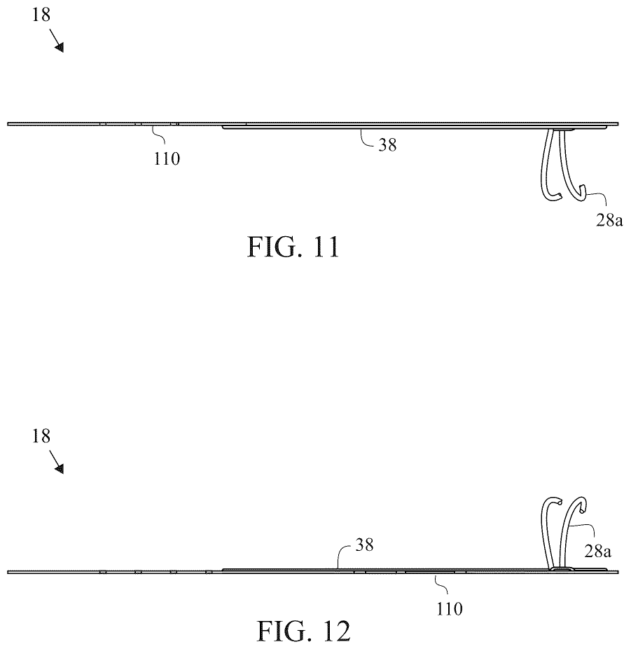

A multifunction electrode pad with integrated 12-lead ECG acquisition apparatus, herein referenced as device 20, comprises of a sternum pad 18 and an apex pad 16. The sternum pad 18, shown in FIGS. 7-12, comprises a body 110 with a first defibrillation pad 30a, a first electrode 115a, a second electrode 115b, and a third electrode 115c. The sternum pad 18 also comprises a right arm (RA) member 27c having a fourth electrode 115d and a line 28a to the body. The apex pad 16, shown in FIGS. 1-6, comprises a body 110 with a second defibrillation pad 30b, a fifth electrode 115e, a sixth electrode 115f, a seventh electrode 115g and an eighth electrode 115h, the apex pad 16 further comprises a left arm (LA) member 27h having a ninth electrode 115i and a lower left (LL) member 27i having a tenth electrode 115j.

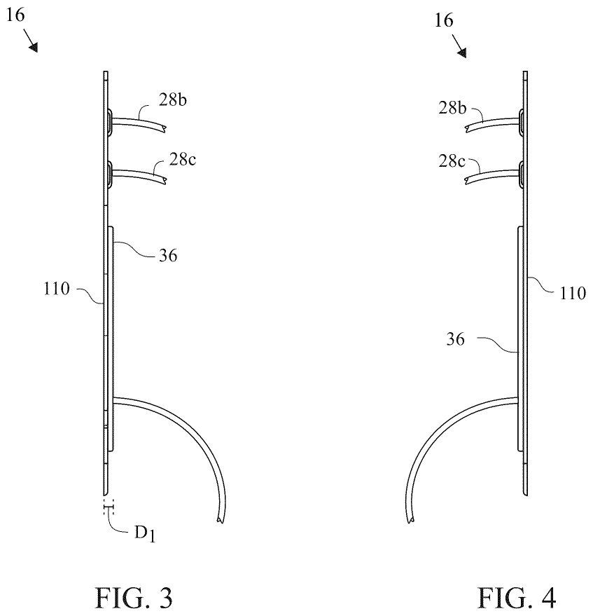

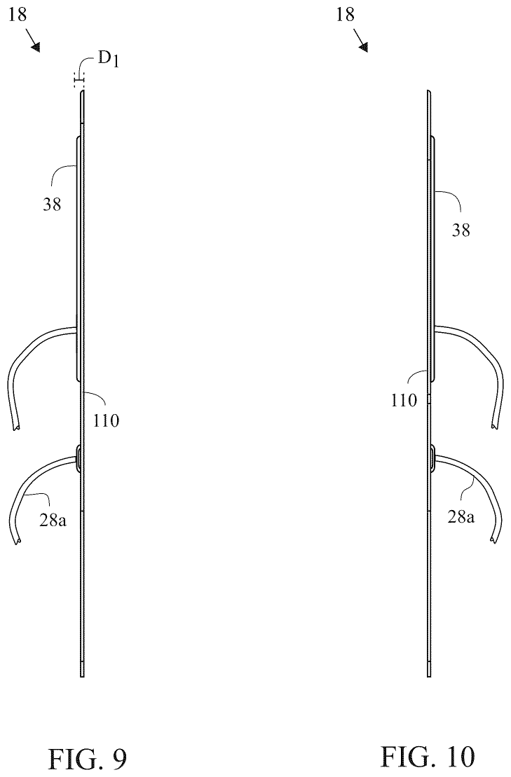

The defibrillation pads 30a-30b are ubiquitous and made of electrically conductive plates 36, 38 in a foam tape substrate 34 with a hydrogel layer 6. The device 20 connects to readily available cables into device specific connectors. Both the sternum pad 18 and the apex pad 16 has a thickness D₁ preferably ranging from 0.3 mm to 1.5 mm, more preferably 0.05 mm to 1.0 mm, and most preferably 0.84 mm (in reference to FIG. 3 and FIG. 9).

The body 110 of the sternum pad 18 further comprises a first elongation section 25a comprising at least one electrode member 27 and an expansion section 26. The expansion section 26 has at least one concertina member 75 integrated with at least one connector member 76. The first electrode 115a (V1) and the second electrode 115b (V2) are positioned on the elongation section 25a.

The body 110 of the apex pad 16 comprises a second elongation section 25b comprising an expansion section 26 comprising at least one electrode member 27, at least one concertina member 75 integrated with at least one connector member 76. The fifth electrode 115e (V3), the sixth electrode 115f (V4), the seventh electrode 115g (V5) and the eighth electrode 115h (V6) are positioned on the second elongation section 25b

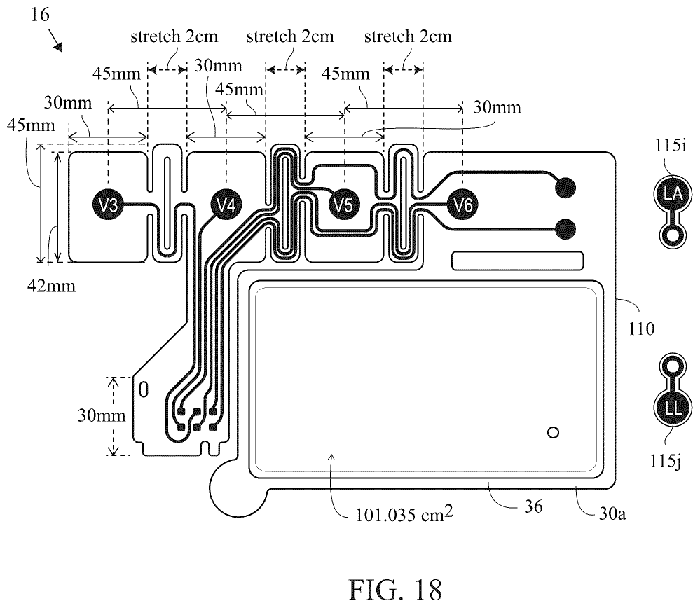

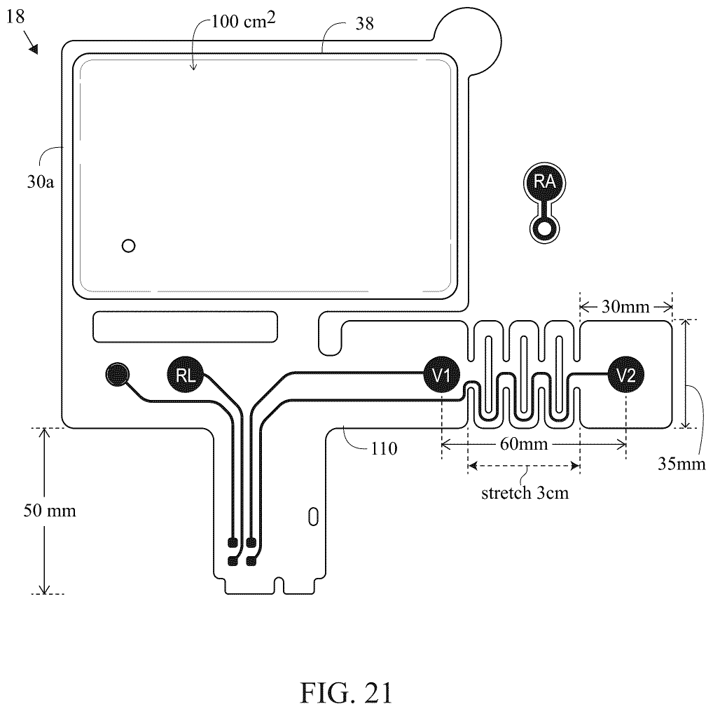

Each of the defibrillation pads 30a, 30b is compatible with standard voltage defibrillations delivery per guidelines (1-360 Joules) and pacemaker voltage delivery (10-140 milliAmps). Each of the defibrillation pads 30a, 30b has a defibrillation electrode pad conductor 38, 36, respectively, with a surface area ranging from 80 cm3 to 90 cm3 (FIG. 18 and FIG. 21). More preferably, the surface has a minimum area of 85 cm³.

Each of the electrodes 115 has a diameter of 8-10 mm and is composed of AgCl material 2 and is surrounded by an adhesive material in a foam 34.

Preferably, the ECG printed circuits 5 of the device 20 have a preferable width of 1-2 mm.

The device 20 is configured to cardiac pace for at least eight hours at 100 milli-Amps at eighty beats per minute.

Another embodiment of the device 20 is a multifunction electrode pad with integrated 12-lead ECG acquisition comprising of a sternum pad 18 and an apex pad 16. The sternum pad 18 comprises a body 110 with a first defibrillation pad 30a and a first elongation section 25a. The elongation section 25a comprises a first electrode member 27a with a first electrode 115a (V1), a second electrode member 27b with a second electrode 115b (V2), an expansion section 26a between the first electrode member 27a and the second electrode member 27b, and a third electrode 115c (RL). The sternum pad 18 also has a right arm member 27c with a fourth electrode 115d (RA) and a line 28a to the body.

The apex pad 16 comprises a body 110 with a second defibrillation pad 30b and a second elongation section 25b. The elongation section 25b comprises a third electrode member 27d with a fifth electrode 115e (V3), a fourth electrode member 27e with a sixth electrode 115f (V4), an expansion section 26b between the third electrode member 27d and the fourth electrode member 27e, a fifth electrode member 27f with a seventh electrode 115g (V5), another expansion section 26c between the fourth electrode member 27e and the fifth electrode member 27f, a sixth electrode member 27g with an eighth electrode 115h (V6), and another expansion section 26d between the fifth electrode member 27f and the sixth electrode member 27g. The apex pad 16 also has a left arm member 27h having a ninth electrode 115i (LA) with a second line 28b to the body, and a lower left member 27i having a tenth electrode 115j (LL) and a third line 28c to the body.

Each of the expansion sections 26a-26d comprises at least one concertina member 75 integrated with at least one connector member 76.

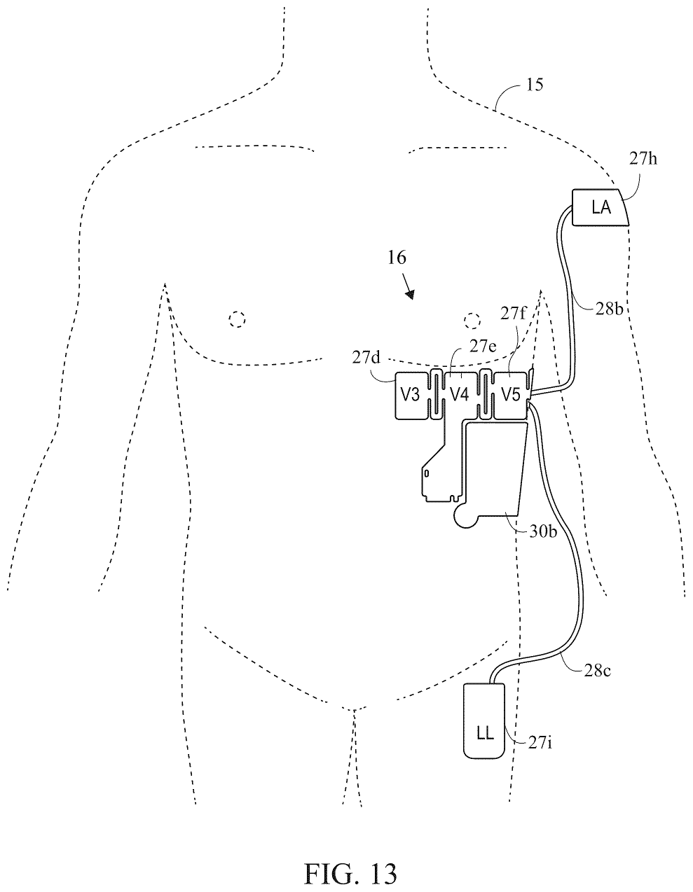

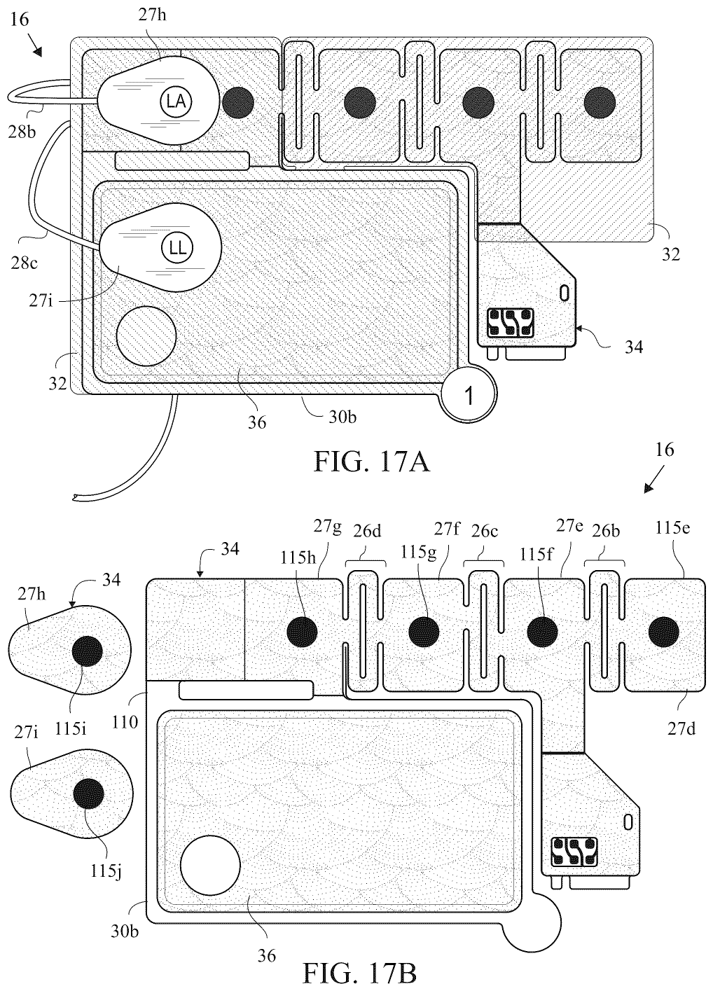

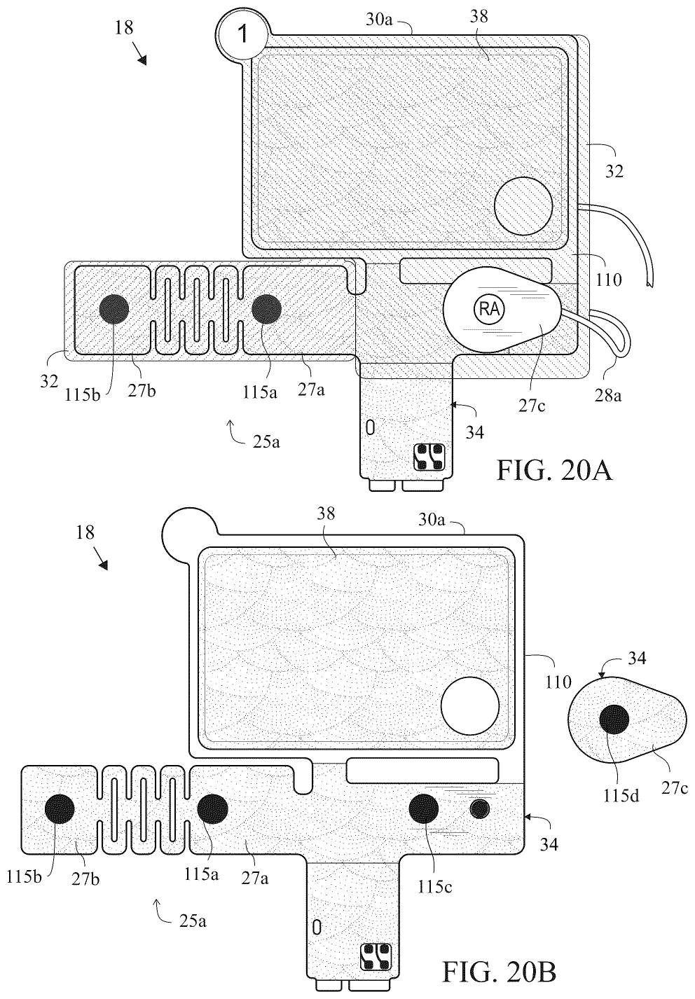

In one embodiment, the RA 115d, LA 115i, and LL 115j electrodes are on the back of the liner 32 (shown in FIGS. 17A, 20A), detaches from the pads 16, 18, and are applied to the proper locations (shown in FIGS. 13-15). The limb lead wire 28a-28c length is preferably 33 cm edge to edge (38 cm total including strips).

The device 20 is preferably composed of hydrogel, metal composites, an adhesive foam body, a release liner, lead wires, connector cables, a 12-lead EKG molded connector, and a defibrillation molded connector. The molded connectors are preferably, clearly labeled. The device 20 is preferably shielded and insulated.

The ECG electrodes are preferably 8-10 mm in diameter of silver chloride, AgCl. The ECG electrodes are covered with hydrogel in diameter of 10-25 mm with a preferred thickness. The ECG electrodes are preferably surrounded by adhesive material in a foam, or other soft substrate typical of defibrillation pads and other ECG electrodes. The ECG electrodes are isolated from the defibrillation electrode pads 16, 18 physically by insulating material, preventing inadvertent conduction of defibrillation.

The ECG printed circuits are preferably 1-2 mm in width. The ECG electrodes and circuits are made of flexible conductive inks that prevent fracture during chest compressions. The ECG printed circuits have resistance of less than 2 ohms.

The defibrillation electrode pad conductor 38, 36 surface preferably has a minimum area of 85 cm3. The defibrillation pad 30a-30b preferably measures at least 8.5 cm by 10 cm. The defibrillation pads 30a-30b preferably are compatible with standard voltage defibrillations delivery per guidelines and pacemaker voltage delivery.

The device 20 is able to deliver fifty defibrillation shocks per use. The device is able to cardiac pace for at least 8 hours at 100 mA at 80 BPM. The ECG electrodes will be compliant with above guidelines and do not draw away defibrillation energy outside of guideline requirements.

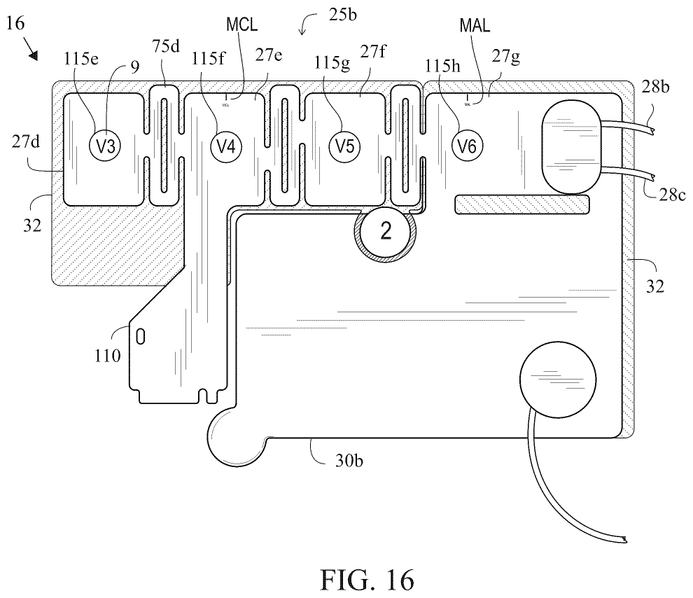

FIGS. 1A-6 illustrate the apex pad 16 from six sides. FIGS. 16-18 illustrate the apex pad 16 with the release liners 32 and foam 34. FIG. 16 shows the apex pad 16 front on liner 32. The tab of liner 2 is labelled and overhanging the defibrillator pad 30b. FIG. 17A shows the apex pad 16 rear on liner 32. The limb lead electrodes, LA 115i and LL 115j, are on the back of the liner 32. FIG. 17B shows the apex pad 16 rear without the liner 32 to expose the portions with foam 34, which is indicated by the pattern. FIG. 18 shows the apex pad 16 front with electrode 115 spacing and track layout.

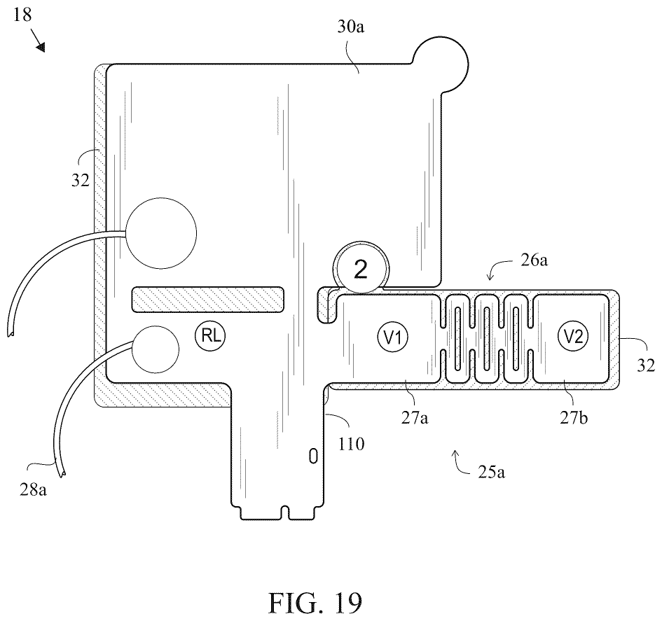

FIGS. 7-12 illustrate the sternum pad 18 and FIGS. 19-21 illustrate the sternum pad 18 with the release liners 32 and foam 34. FIG. 19 shows the sternum pad 18 front on liner 32. The tab of liner 2 is labelled and overhanging the defibrillator pad 30a. FIG. 20A shows the sternum pad 18 rear on liner 32. The limb lead electrode RA 115d is on the back of the liner 32. FIG. 20B shows the sternum pad 18 rear without the liner 32 to expose the portions with foam 34, which is indicated by the pattern. FIG. 21 shows the sternum pad 18 front with electrode 115 spacing and track layout.

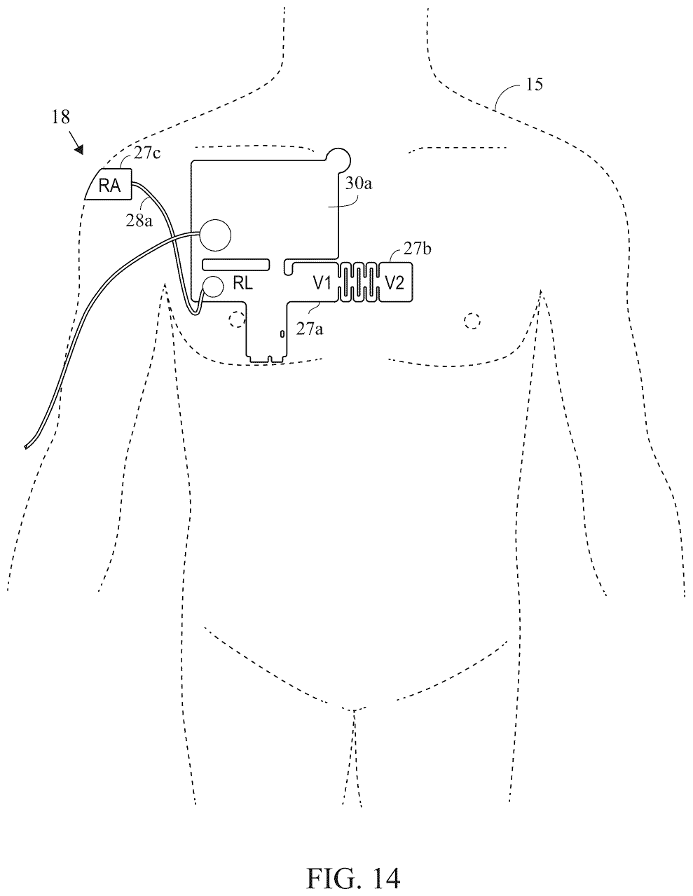

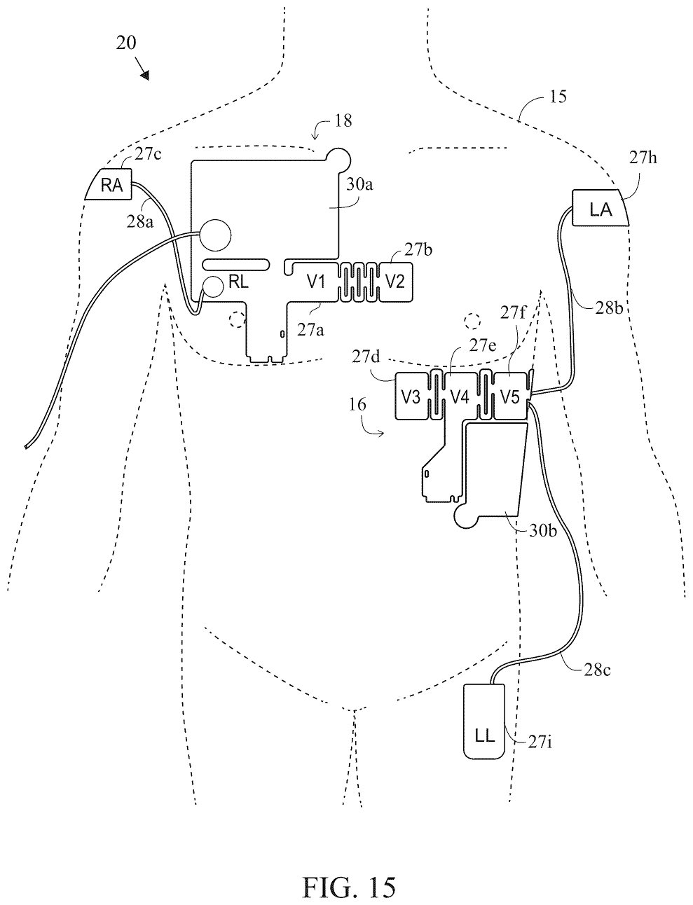

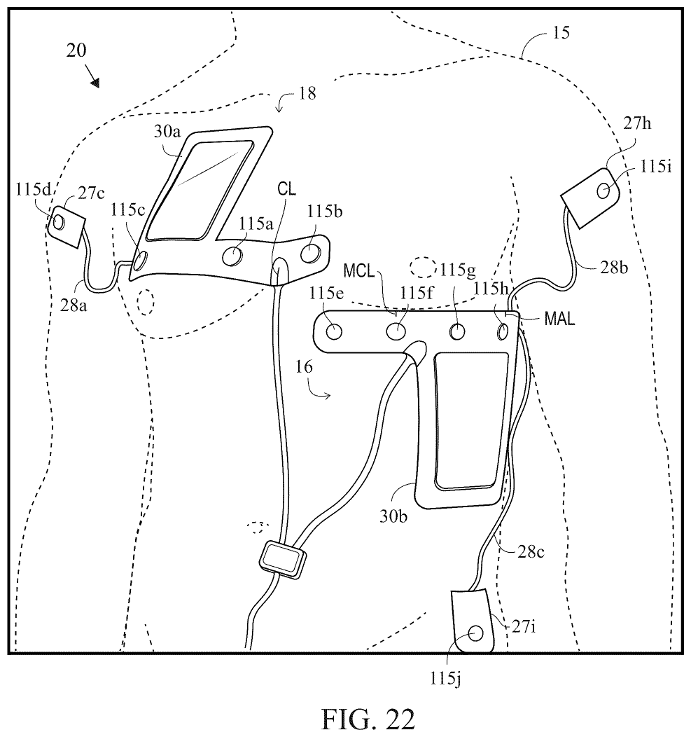

FIGS. 13-15 shows a user 15 wearing the device 20. In FIG. 13, the apex pad 16 is applied in the appropriate position with limb leads 27h, 27i LA and LL and lead wires 28b, 28c extending out from the apex pad 16. FIG. 14 shows the sternum pad 18 applied in the appropriate position with limb lead 27c RA and lead wire 28a extending out from the sternum pad 18. The remaining lead wire connects to a defibrillator/cardiac monitor (shown in FIG. 1A and FIG. 7). FIG. 15 shows the device 20 with both pads 16, 18 applied. FIG. 22 shows another embodiment of the device 20 applied to a user 15.

The body 110 has a plurality of contacts 4 positioned on an end portion of the center section of the body 110. Each of the contacts 4 are preferably composed of an abrasive resistant ink. Each of the extension members are preferably configured for at least double length extension from an un-extended state, alternatively 50% extension from an un-extended state, and preferably at least a 25% extension from an un-extended state. The body 110 preferably comprises a layer of an unwoven fabric. Each of the printed wires 5 and each of the printed electrodes 115 are preferably composed of a printable conductive silver.

Each concertina member 75 is preferably rectangular in shape with a central aperture, allowing for extension of the extension member 25. Each concertina member 75 preferably has a first side panel, a second side panel parallel to the first side panel, a third side panel perpendicular and connecting to first ends of the first side panel and the second side panel, and a fourth side panel parallel to the third side panel and connecting to the second ends of the first side panel and the second side panel. The first side panel, the second side panel, the third side panel and the fourth side panel define the central aperture.

Those skilled in the pertinent are will recognize that the extension members may have alternative numbers of expansion sections and electrode sections, and alternative numbers of concertina members and connector members without departing from the scope and spirit of the present invention.

The electrodes 115 are preferably screen-printed, comprised of ten electrodes indexed to meet American Heart Association (AHA) guidelines for diagnostic criteria 12-lead ECG and additional node positions for diagnostic studies for right sided interpretation and posterior interpretation lead positioning.

The device 20 in one embodiment comprises of placement markers 9, a polyester layer, wired circuits (printed silver tracing) 5, carbon contacts 4, an unwoven fabric layer, an AgCl components layer, an adhesive layer, a hydrogel components layer, and a backing liner layer. A dielectric layer is not shown.

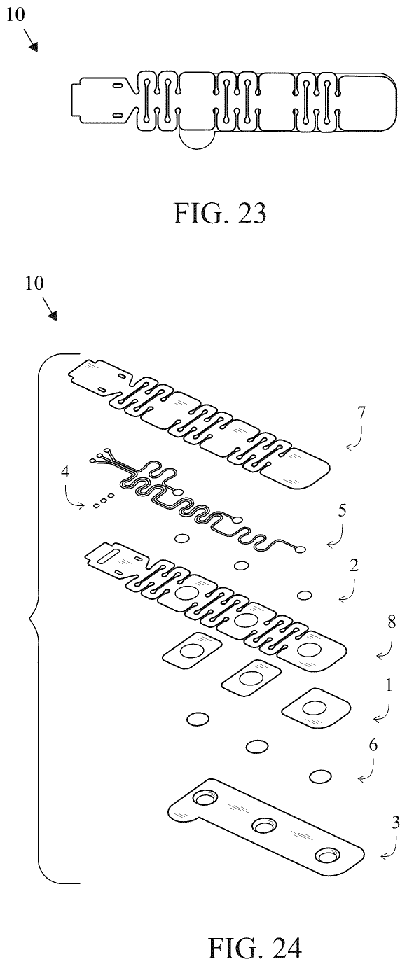

A 3-lead component 10, shown in FIGS. 23-24, allows for the 12-Lead ECG device to expand to a 15-lead ECG device. The 3-lead component 10 comprises of a polyester layer 7, wired circuits (printed silver tracing) 5, carbon contacts, an AgCl components layer 2, an unwoven fabric layer 8, an adhesive layer 1, a hydrogel components layer 6, and a backing liner layer 3. A dielectric layer is not shown.

An electrode allows for the acquisition of superficial electrical activity.

A wireless electrode interface carries the electrical activity to a transmitter or device directly.

A powered transmitter is a long-life Battery Powered Wireless analog-to-analog or analog-to-digital transmission with or without amplification, or alternatively, a direct powered connection between transmitter and receiver with or without amplification through a direct machine connection.

A powered receiver is a long-life Battery Powered Wireless analog-to-analog or digital-analog receiver with or without amplification.

A direct wired connector is a wire to an ECG machine interface, a multi-pin connector with or without amplification.

A conductive elastic rubber material is disclosed in U.S. Pat. No. 8,491,884, which pertinent parts are hereby incorporated by reference.

A stretchable graphene film material is disclosed in Chen et al., U.S. Patent Publication Number 20150273737, which pertinent parts are hereby incorporated by reference.

A flexible conductive material comprising silver is disclosed in Taguchi et al., U.S. Patent Publication Number 20130056249, which pertinent parts are hereby incorporated by reference.

The product is intended to be used by trained medical personnel (paramedics, nurses, doctors, etc.) and, if connected to automated external defibrillators (AEDs), by untrained laypersons. The device is intended for single patient use, is disposable, is sold non-sterile, and is packaged in a hermetically sealed pouch.

The device is available in 3 forms: Adult Composite 12-lead Multi-Function Defibrillation Electrode Pad; Pediatric Composite 12-lead Multi-Function Defibrillation Electrode Pad; and Infant Composite 12-lead Multi-Function Defibrillation Electrode Pad.

In preferred embodiments:

The device preferably adheres to a patient for at least 8 hours.

The device packaging will be marked with specific tear-open locations.

The device labeling will clearly demonstrate proper positioning of the multifunction pads and proper positioning of the cardiac ECG electrodes.

The device labeling preferably includes a marker for V4 with mid clavicular notation, a marker for V6 with mid axillary notation, and markers for placement of V1 and V2.

The device preferably clearly labels the extremity locations with RA for right upper extremity, LA for left upper extremity, and LL for left lower extremity. Alternatively, the device shall clearly label the extremity locations with RUE for right upper extremity, LUE for left upper extremity, and LLE for left lower extremity.

Preferably, the device can remain in place during cardiopulmonary resuscitation (CPR)/chest compressions for up to 30 minutes. The device electrode cables and printed wires preferably can tolerate chest compressions/CPR for up to 30 minutes.

The adult device preferably accommodates 5^{th} percentile to 95^{th} percentile sized adult patients. The infant device is intended for infants and children under 1 years of age. The pediatric device is intended for pediatric patients.

The device is preferably compliant with ANSI AAMI EC11, EC12, EC53 and IEC 60601-2-4. The device is preferably compliant to ISO10993 biocompatibility standards and ISTA2 standards.

The device can preferably be stored at 15-35 degrees Celsius and have an unopened shelf life of 24 months.

Dunphy et al., U.S. Pat. No. 9,986,929 for an Emergency Cardiac And Electrocardiogram Electrode Placement System is hereby incorporated by reference in its entirety.

Dunphy et al., U.S. Pat. No. 10,893,818 for an Emergency Cardiac And Electrocardiogram Electrode Placement System is hereby incorporated by reference in its entirety.

Dunphy et al., U.S. Pat. No. D872279 for an Emergency Cardiac And Electrocardiogram Electrode Placement System is hereby incorporated by reference in its entirety.

Ronan et al., U.S. Pat. No. D877912, for a Cable Controller For An Electrocardiogram Electrode Placement System is hereby incorporated by reference in its entirety.

McClung et al., U.S. Pat. No. 11,701,048, for an Emergency Cardiac And Electrocardiogram Electrode Placement System With Artificial Intelligence is hereby incorporated by reference in its entirety.

McClung et al., U.S. Pat. No. 11,864,858, for an Emergency Cardiac And Electrocardiogram Electrode Placement System With Wireless Electrodes is hereby incorporated by reference in its entirety.

McClung et al., U.S. Pat. No. 11,896,393, for a Wearable Diagnostic Electrocardiogram Garment is hereby incorporated by reference in its entirety.

McClung et al., U.S. Patent Publication Number 2022017592 (U.S. patent application Ser. No. 17/665,003, filed on Mar. 8, 2020) for a Screen Printed Electrodes For An Electrocardiogram Article is hereby incorporated by reference in its entirety.

McClung et al., U.S. patent application Ser. No. 18/384,872, filed on Oct. 29, 2023, for an Electrocardiogram Apparatus is hereby incorporated by reference in its entirety.

From the foregoing it is believed that those skilled in the pertinent art will recognize the meritorious advancement of this invention and will readily understand that while the present invention has been described in association with a preferred embodiment thereof, and other embodiments illustrated in the accompanying drawings, numerous changes modification and substitutions of equivalents may be made therein without departing from the spirit and scope of this invention which is intended to be unlimited by the foregoing except as may appear in the following appended claim. Therefore, the embodiments of the invention in which an exclusive property or privilege is claimed are defined in the following appended claims.

We claim as our invention the following:

1. A multifunction electrode pad with integrated 12-Lead ECG acquisition apparatus comprising:
a sternum pad comprising a body with a first defibrillation pad, a first electrode, a second electrode, and a third electrode, the sternum pad also comprising a right arm member having a fourth electrode and a line to the body;
an apex pad comprising a body with a second defibrillation pad, a fifth electrode, a sixth electrode, a seventh electrode and an eighth electrode, the apex pad also comprising a left arm member having a ninth electrode and a lower left member having a tenth electrode;
wherein each of the sternum pad and the apex pad is composed of an electrical conductive plates in a foam tape substrate with a hydrogel layer.

2. The apparatus according to claim 1 wherein the body of the sternum pad further comprises a first elongation section comprising at least one electrode member, an expansion section comprising at least one concertina member integrated with at least one connector member, wherein the first electrode and the second electrode are positioned on the first elongation section, and wherein the body of the apex section comprises a second elongation section comprising an expansion section comprising at least one electrode member, at least one concertina member integrated with at least one connector member, wherein the fifth electrode, the sixth electrode, the seventh electrode and the eighth electrode are positioned on the second elongation section.

3. The apparatus according to claim 1 wherein each of the first defibrillation pad and the second defibrillation pad is compatible with standard voltage defibrillations delivery per guidelines of 1-360 Joules, and pacemaker voltage delivery of 10-140 milliAmps.

4. The apparatus according to claim 1 wherein each of first electrode, the second electrode, the third electrode, the fourth electrode, the fifth electrode, the sixth electrode, the seventh electrode, the eighth electrode, the ninth electrode and the tenth electrode has a diameter of 8-10 mm and is composed of AgCl material.

5. The apparatus according to claim 4 wherein each of first electrode, the second electrode, the third electrode, the fourth electrode, the fifth electrode, the sixth electrode, the seventh electrode, the eighth electrode, the ninth electrode and the tenth electrode is surrounded by an adhesive material in a foam.

6. The apparatus according to claim 5 further comprising ECG printed circuits with a 1-2 mm width.

7. The apparatus according to claim 1 wherein each of the sternum pad and the apex pad has defibrillation electrode pad conductor surface has an area ranging from 80 cm³ to 90 cm³.

8. The apparatus according to claim 1 wherein each of the sternum pad and the apex pad has a defibrillation electrode pad conductor surface with a minimum area of 85 cm³.

9. The apparatus according to claim 1 wherein each of sternum pad and the apex pad has a thickness ranging from 0.3 mm to 1.5 mm.

10. The apparatus according to claim 1 wherein the apparatus is configured to cardiac pace for at least eight hours at 100 milli-Amps at eighty beats per minute.

11. A multifunction electrode pad with integrated 12-Lead ECG acquisition apparatus comprising:
a sternum pad comprising a body with a first defibrillation pad and a first elongation section, the first elongation section comprising a first electrode member with a first electrode, a second electrode member with a second electrode, a first expansion section between the first electrode member and the second electrode member, and a third electrode, the sternum pad also comprising a right arm member having a fourth electrode and a first line to the body;
an apex pad comprising a body with a second defibrillation pad and a second elongation section, the second elongation section comprising a third electrode member with a fifth electrode, a fourth electrode member with a sixth electrode, a second expansion section between the third electrode member and the fourth electrode member, a fifth electrode member with a seventh electrode, a third expansion section between the fourth electrode member and the fifth electrode member, a sixth electrode member with an eighth electrode, a fourth expansion section between the fifth electrode member and the sixth electrode member, the apex pad also comprising a left arm member having a ninth electrode with a second line to the body, and a lower left member having a tenth electrode and a third line to the body;
wherein each of the sternum pad and the apex pad is composed of an electrical conductive plates in a foam tape substrate with a hydrogel layer.

12. The apparatus according to claim 11 wherein each of the first expansion section, second expansion section, the third expansion section and the fourth expansion section comprises at least one concertina member integrated with at least one connector member.

13. The apparatus according to claim 11 wherein each of first electrode, the second electrode, the third electrode, the fourth electrode, the fifth electrode, the sixth electrode, the seventh electrode, the eighth electrode, the ninth electrode and the tenth electrode has a diameter of 8-10 mm and is composed of AgCl material.

14. The apparatus according to claim 13 wherein each of first electrode, the second electrode, the third electrode, the fourth electrode, the fifth electrode, the sixth electrode, the seventh electrode, the eighth electrode, the ninth electrode and the tenth electrode is surrounded by an adhesive material in a foam.

15. The apparatus according to claim 14 further comprising ECG printed circuits with a 1-2 mm width.

16. The apparatus according to claim 11 wherein each of the sternum pad and the apex pad has defibrillation electrode pad conductor surface has an area ranging from 80 cm³ to 90 cm³.

17. The apparatus according to claim 11 wherein each of the sternum pad and the apex pad has a defibrillation electrode pad conductor surface with a minimum area of 85 cm³.

18. The apparatus according to claim 11 wherein each of sternum pad and the apex pad has a thickness ranging from 0.3 mm to 1.5 mm.
